(19) United States
(12) Patent Application Publication
(10) Pub. No.: US 2026/0354692 A1
(43) Pub. Date: Nov. 12, 2026

(54) METHOD OF MEASURING, PROCESSING AND ANALYZING THE PHOTOPLETHYSMOGRAPHIC SIGNAL FOR THE NON-INVASIVE DETERMINATION OF THE FILLING PRESSURE OF THE LEFT VENTRICLE OF THE HEART AND A DEVICE FOR PERFORMING THIS METHOD

(71) Applicant: Seerlinq S.R.O., Trnava (SK)

(72) Inventors: Allan BÖHM, Bratislava (SK); Branislav Bezák, Bratislava (SK); Nikola Jajcay, Svätý Jur (SK)

(21) Appl. No.: 19/715,578

(22) Filed: Jun. 22, 2026

Related U.S. Application Data
(63) Continuation of application No. PCT/SK2024/000007, filed on Dec. 20, 2024.
(63) Continuation of application No. PCT/SK2024/000004, filed on Jul. 31, 2024.

(30) Foreign Application Priority Data
Dec. 22, 2023 (SK) PP 162-2023
Dec. 22, 2023 (SK) PUV 221-2023

(51) Int. Cl. A61B 5/021; A61B 5/00; A61B 5/024

(52) U.S. Cl. CPC A61B 5/02108; A61B 5/024; A61B 5/7207; A61B 5/7221; A61B 5/742

(57) ABSTRACT

A method of measuring, processing and analyzing the photoplethysmographic signal for the non-invasive determination of the filling pressure of the left ventricle of the heart using a sensor (1) of the photoplethysmographic signal connected or connectable to a device containing a processor for executing program instructions for processing the sensed photoplethysmographic signal and a user interface for displaying or interpreting the results of the processing and analyzing the photoplethysmographic signal via a user interface, where the photoplethysmographic signal is measured when the user's position changes, while this signal is processed to analyze the parameters of the photoplethysmographic signal curve by statistical methods or advanced machine learning methods, which classify the change in the parameters of the photoplethysmographic signal when the user's position changes.

### CROSS-REFERENCE TO RELATED APPLICATIONS

This application is a continuation of PCT Application No. PCT/SK2024/000007, filed on Dec. 20, 2024, and PCT Application No. PCT/SK2024/000004, filed Jul. 31, 2024, each of which claims priority to Slovakian Priority Application No. PP 162-2023, filed Dec. 22, 2023, and Slovakian Priority Application No. PUV 221-2023 filed Dec. 22, 2023, the contents of each of which are incorporated herein by reference in their entireties.

### TECHNICAL FIELD

The invention relates to a method of measuring, processing and analyzing the photoplethysmographic signal for the non-invasive determination of the filling pressure of the left ventricle of the heart and a device for performing this method.

### BACKGROUND

0.4-2.2% of the population in developed countries suffer from heart failure (HF)-more than 10 million patients in the EU and 6 million in the USA. 0.5-0.6 million cases are diagnosed each year with increasing incidence. As a result of the frequent hospitalizations of these patients, 1-2% of total health care expenditure is spent on HF. It is expected that the costs of HF in the USA alone reach at least USD 160 billion per year by 2030. Patients hospitalized with HF have a 50% risk of rehospitalization within 6 months, HF symptoms recur in 65% of patients within 3 months, and patients are limited by symptoms that significantly impair their quality of life.

The concept of telemonitoring for patients with HF was introduced with the aim of reducing the number of decompensations and hospitalizations for HF, reduce mortality, total costs per patient, improve treatment personalization, patient quality of life and efficiency for an overburdened healthcare system.

The first telemonitoring systems were based on monitoring weight, vital functions and symptoms perceived by patients, for example through telephone checks. Although results from smaller studies have been promising, this approach has failed to demonstrate benefits in large, randomized trials, Stevenson, Lynne Warner, et al. “Remote Monitoring for Heart Failure Management at Home.” Journal of the American College of Cardiology 81.23 (2023): 2272-2291. However, a recent meta-analysis of 29 studies with 10,981 patients showed a modest but statistically significant improvement in quality of life and a reduction in hospitalizations for patients with HF, Takeda A, Martin N, Taylor RS, et al. Disease management interventions for heart failure. Cochrane Database Syst Rev. 2019; 1:CD002752.s. On the other hand, symptoms in these patients are often not recognized until shortly before hospitalization, and even patients who perceive this change are often unsure of the meaning of the symptoms and delay seeking medical care, Altice NF, Madigan EA. Factors associated with delayed care-seeking in hospitalized patients with heart failure. Heart Lung. 2012; 41:244-254. Additionally, weight measurement has been shown to be a poor signal with significant variation due to bowel habits and caloric intake with very little sensitivity (10 to 20 %) for detecting worsening HF condition, Adamson PB. Pathophysiology of the transition from chronic compensated and acute decompensated heart failure: new insights from continuous monitoring devices. Curr Heart Fail Rep. 2009; 6(4): 287-292. Several digital health care systems that combine weight with other variables such as heart rate, blood pressure, patient-observed symptoms or exercise data, are currently in clinical trials, Stevenson, Lynne Warner, et al. “Remote Monitoring for Heart Failure Management at Home.” Journal of the American College of Cardiology 81.23(2023): 2272-2291.

Although symptoms of HF, such as edema or dyspnea, develop relatively suddenly, they are preceded by complex pathophysiological processes. These include progressive increases in cardiac filling pressures, neurohormonal and hemodynamic changes, and volume redistribution.

Several different monitoring systems based on monitoring these pathophysiological changes have been developed and studied with the aim of improving outcomes.

Pulmonary congestion (accumulation of fluid in the lung tissue) is the cause of one of the most significant symptoms of heart failure. Several methods are currently available to measure the level of pulmonary congestion. Most lung tissue is filled with air, which is a poor electrical conductor. The accumulation of conductive fluid in the lungs leads to a decrease in electrical impedance. Modern implantable for HF patients (CRT—cardioresynchronization therapy, ICD—implantable cardioverter-defibrillator, CRTD—cardiac resynchronization therapy with defibrillator) can monitor changes in electrical impedance between the lead electrodes in the heart and the pulse generator on the chest wall. These changes in bioimpedance can be detected as early as 2 to 3 weeks before hospitalization of a patient with HF with a return to the baseline value usually a week or more after discharge. An example of such a solution is CorVue from St. Jude Medical (St. Paul, MN, USA) or OptiVol from Medtronic (Minneapolis, MN, USA). A correlation between reduced thoracic impedance and worsening of HF symptoms has been demonstrated in several small trials. However, a meta-analysis and meta-regression of several randomized trials did not demonstrate an overall benefit compared to usual management, Hajduczok AG, Muallem SN, Nudy MS, et al. Letter to the editor to update the article “Remote monitoring for heart failure using implantable devices: a systematic review, meta-analysis, and meta-regression of randomized controlled trials”. Heart Fail Rev. 2022; 27(3):985-987.

Multiple trials, including REM-HF, Morgan JM, Kitt S, Gill J, et al. Remote management of heart failure using implantable electronic devices. Eur Heart J. 2017; 38:2352-2360, CLEPSYDRA, Auricchio A, Gold MR, Brugada J, et al. Long-term effectiveness of the combined minute ventilation and patient activity sensors as predictor of heart failure events: the CLEPSYDRA study. Eur J Heart Fail. 2014; 16:663-670, MultiSENSE Trial, Boehmer JP, Hariharan R, Devecchi FG, et al. A multisensor algorithm predicts heart failure events in patients with implanted devices: results from the MultiSENSE study a Gardner RS, Singh JP, Stancak B, et al. Heart-Logic multisensor algorithm identifies patients during periods of significantly increased risk of heart failure events: results from the MultiSENSE study. Circ Heart Fail. 2018; 11(7):e004669. J Am Coll Cardiol HF. 2017; 5:216-225, SELENE-HF, D'Onofrio A, Solimene F, Calo L, et al. Combining home monitoring temporal trends from implanted defibrillators and baseline patient risk profile to predict heart failure hospitalizations: results from the SELENE HF study. Europace. 2022; 24:234-244, or TRIAGE-HF, Virani SA, Sharma V, McCann M, et al. Prospective evaluation of integrated device diagnostics for heart failure management: results of the TRIAGE-HF study. ESC Heart Fail. 2018; 5:809-817 a Adamson, Philip B. “Pathophysiology of the transition from chronic compensated and acute decompensated heart failure: new insights from continuous monitoring devices.” Current heart failure reports 6.4(2009): 287-292, investigated the possibilities of improving the diagnostic properties of pulmonary impedance measurements by combining them with other physiological parameters such as heart rate, heart rate variability, activity or heart sounds. For example, the MultiSENSE trial, investigated the HeartLogic monitoring system from Boston Scientific (Marlborough, MA, USA) a CRTD-based monitoring system with impedance measurement option and monitoring of heart and respiratory rate, activity and heart echoes, demonstrated a sensitivity of 70% and specificity of 85.7% for forecasting worsening of HF, with a median of 34 days from alert to event. On the other hand, the REM-HF trial investigating the clinical and cost effectiveness of remote monitoring of patients with CRT/CRT-D or ICD using HF risk scores based on chest impedance, activity, and heart rate (nocturnal heart rate, rhythm, and heart rate variability) did not prove any effect on mortality from any cause or unplanned hospitalizations in patients with HF.

Thoracic impedance can also be used to estimate left ventricular ejection volume and filling pressures of the heart based on blood volume changes in the chest. However, this method, called impedance cardiography, has shown only a moderate and weak correlation with cardiac output and pulmonary artery pressures and did not correlate with subsequent death or hospitalization within 6 months, Kamath SA, Drazer MH, Tassisa, et al. Correlation of impedance cardiography with invasive hemodynamic measurements in patients with advanced heart failure: the bioimpedance cardiography (BIG) sub study of the ESCAPE trial. Am Heart J. 2009; 158:217-223.

A further method for assessing the level of pulmonary congestion is remote dielectric sensing (ReDS) based on the analysis of a focused electromagnetic signal with a transmitter and sensor built into the chest belt. This technology has been approved by the Food and Drug Administration (FDA) and has also received European CE certification for monitoring the amount of fluid in the lungs. ReDS correlated with pulmonary wedge pressure, was able to detect pulmonary edema and accurately differentiate patients with HF and pulmonary congestion (defined by chest computed tomography—CT) from normal individuals. The SMILE randomized trial of 268 patients showed a 48% reduction in hospitalizations of patients with HF during a 6-month follow-up, Abraham WT, Anker S, Burkhoff D, et al. Primary results of the sensible medical innovations lung fluid status monitor allow reducing readmission rate of heart failure patients (SMILE) trial. J Card Fail. 2019; 25(11):938.

The MicroCore system from ZOLL Medical (Chelmsfor, MA, USA) is a radiofrequency sensor patch that is applied to the lateral chest wall. Although data are very limited, the BMAD trial demonstrated a reduction in the number of hospitalizations of patients with HF as well as in the composite outcome-reduction in hospitalizations of patients with HF, deaths, and emergency department visits, Boehmer J, et al. Impact of Heart Failure Management Using Thoracic Fluid Monitoring From a Novel Wearable Sensor: Results of the Benefits of Microcor (μCor™) in Ambulatory Decompensated Heart Failure (BMAD) Trial. Presented as Late Breaking Clinical Trial at the 2023 American College of Cardiology Annual Scientific Session, Mar. 6, 2023.

A smaller trial Amir, Offer, et al. “Remote speech analysis in the evaluation of hospitalized patients with acute decompensated heart failure.” Heart Failure 10.1 (2022): 41-49 in 40 adult patients demonstrated that the level of congestion can also be measured by analysis voice changes using automated speech analysis technology. This methodology is based on the effect of changes in lung volumes and soft tissue edema of the vocal tract during congestion on the spectral parameters of speech.

A further option for monitoring HF status is based on direct invasive measurement of cardiac pressures. Cardiac filling pressures have been shown to rise steadily 3 weeks prior to hospitalization for HF in patients with HF with preserved ejection fraction (HFpEF) and 4 weeks before hospitalization for HF with reduced ejection fraction (HFrEF), Zile, Michael R., et al. “Transition from chronic compensated to acute decompensated heart failure: pathophysiological insights obtained from continuous monitoring of intracardiac pressures.” Circulation 118.14 (2008): 1433-1441.

CardioMEMS is a pressure monitoring sensor implanted in a branch of the pulmonary artery (PA) developed by Abbott (Abbott Park, IL, USA). The randomized CHAMPION trial demonstrated a 37% reduction in hospitalizations for HF, Abraham, William T., et al. “Wireless pulmonary artery haemodynamic monitoring in chronic heart failure: a randomised controlled trial.” The Lancet 377.9766 (2011 ): 658-666. GUIDE-HF trial approved by the US Food and Drug Administration (FDA) showed a significant reduction in hospitalizations for HF that was also comparable between patients with HFrEF and HFpEF, Zile, Michael R., et al. “Hemodynamically-guided management of heart failure across the ejection fraction spectrum: the GUIDE-HF trial.” Heart Failure 10.12 (2022 ): 931-944. Further trials have demonstrated benefit for reducing PA pressure and hospitalizations for HF at 6, 12, and 24 months, Stevenson, Lynne Warner, et al. “Remote Monitoring for Heart Failure Management at Home.” Journal of the American College of Cardiology 81.23(2023 ): 2272-2291.

The HeartPod system developed by Savacor, later acquired by St. Jude Medical (St. Paul, MN, USA) was a small implantable device capable of monitoring left atrial (LA) pressures. Although the LAPTOP trial was prematurely terminated due to complications with device insertion through the atrial septum, the analysis of interim results showed a reduction in hospitalizations for HF comparable to the results of the CHAMPION trial, Abraham, William T., et al. “Hemodynamic monitoring in advanced heart failure: results from the LAPTOP-HF trial.” Journal of Cardiac Failure 22.11 (2016 ): 940. In addition, a meta-analysis of the LAPTOP, GUIDE-HF, and CHAMPION trials showed a significant reduction in mortality at two years.

A further device designated for direct LA pressure monitoring called V-LAP from Vectorious Medical Technologies (Tel Aviv, Israel) was successfully implanted into the interatrial septum in 24 patients. There were no device-related complications and LA pressure correlated well with wedge pressure at 3 months, Perl L, Meerkin D, D'Amario D, et al. The V-LAP system for remote left atrial pressure monitoring of patients with heart failure: remote left atrial pressure monitoring. J Cardiac Fail. 2022; 28:963-972.

A study of 33 individuals before planned cardiac catheterization by Silber et al. demonstrated that measurement of the pulse amplitude ratio of the PPG signal during the Valsalva maneuver reflects left ventricular filling pressures, Silber, Harry A., et al. “Finger photoplethysmography during the Valsalva maneuver reflects left ventricular filling pressure.” American Journal of Physiology-Heart and Circulatory Physiology 302.10(2012 ): H2043-H2047. These results were subsequently confirmed by further trials. This system is based on the measurement of changes in cardiac pressure induced by the Valsalva maneuver.

Related to the mentioned system is the document EP 2 706 907 A2, which describes an automated device and a method for non-invasive detection of heart filling pressure. The device includes a computer-readable medium programmed to analyze the pulse amplitude of the photoplethysmographic signal. The analysis is performed for a purpose of determining a point in time when the pulse amplitude of the photoplethysmographic signal does not change by a predetermined value during the first predetermined time period. The method also includes displaying instructions to the user for preparing to initiate forced exhalation against resistance, so-called Valsalva maneuver. This exhalation effort performed by the user is compared to a predetermined resulting range for the exhalation effort, and an indicator of the user's expiratory effort relative to the predetermined resulting range for expiratory effort is displayed on the user interface.

The mentioned solution is based on the use of the so-called Valsalva maneuver, to induce changes in pressures in the chest that cause changes in the circulatory system. However, this maneuver is difficult to reproduce, so users cannot repeat this maneuver correctly. When exhaling forcefully against resistance, the user often shakes, which is reflected in a larger number of artifacts. With this solution, the need for additional equipment to measure expiratory effort arises, with the aim of standardizing measurement. In the event that the Valsalva maneuver cannot be performed, this document describes a device capable of supplying the user with air under pressure.

The aim of the proposed solution is to substantially remedy the shortcomings of the prior art.

### SUMMARY

The mentioned goal is achieved by a method of measuring, processing and analyzing the photoplethysmographic signal, hereinafter referred to as a “PPG signal”, for the non-invasive determination of the filling pressure of the left ventricle of the heart, according to the present invention. The method according to the invention uses a PPG signal sensor connected or connectable to a device containing a processor for executing program instructions for processing and analyzing the sensed PPG signal and a user interface for displaying or interpreting the results of the processing and analyzing the PPG signal via a user interface, or sensor of the PPG signal connected or connectable to a device containing means for transmitting the sensed PPG signal to a remote device containing a processor for executing program instructions for processing and analyzing the PPG signal, means for receiving the results of the processing and analyzing the PPG signal from the remote device containing a processor, and a user interface for displaying or interpreting the results of the processing and analyzing the PPG signal via a user interface.

In the method according to the present invention, the PPG signal is sensed by a PPG signal sensor or a PPG signal sensing device during a passive or active change in the position of the user, which leads to an increased venous return of blood to the heart. The PPG signal is sensed continuously before, during and after changing the user's position, or separately in the first position of the user and separately in the second position of the user after changing his or her position.

The sensed PPG signal is subjected to denoising and filtering to specific frequencies.

The denoised and filtered PPG signal is evaluated using one, more or a combination of signal quality indices selected from the group-asymmetry coefficient, kurtosis, signal-to-noise ratio, entropy, rate of crossings through the zero axis, relative spectral power, peak value of the autocorrelation function, zero crossing of the autocorrelation function, spectral entropy, all of which are calculated on the time series as a whole and then on individual pulses and cumulatively to determine the quality of the photoplethysmographic signal sufficient for the determination of the filling pressure of the left ventricle of the heart.

The PPG signal is processed in the following steps in the mentioned, or in a different order:

Identification of artifacts; identification of individual heart beats; identification of the parts of the curve of the PPG signal to obtain one, more or a combination of parameters selected from the below stated group or groups:

Physiological parameters—blood oxygen saturation, heart rate calculated from individual pulses or from Empirical decomposition into modes, respiratory rate calculated from individual pulses—XB123EF1FM1 algorithm or from empirical decomposition into modes;

Pulse wave characteristics—systolic peak position, systolic peak value, diastolic peak position, diastolic peak value, position of the lowest point on the wave, value of the lowest point on the wave, amplitude (value of the highest point on the wave), position of the highest point on the wave, maximum slope, systolic length expressed in percentiles for percentiles [0.1, 0.25, 0.33, 0.5, 0.66], index B/A, whereby B is the value of the minimum of the second derivative of the PPG signal and A is the value of the maximum of the second derivative of the PPG signal, diastolic length expressed in percentiles for percentiles [0.1, 0.25, 0.33, 0.5, 0.66], the average, minimum and maximum of the NN intervals, the minimum and maximum of the differences in the NN intervals, the standard deviation of the NN intervals, average of the standard deviations of all NN intervals for each 5-minute segment in the long recording, the standard deviation of the averages of NN intervals for each 5-minute segment in a long recording, the root mean square value of the differences in NN intervals, the number of NN intervals over 20, resp. 50 ms, spectral power for very low frequency up to 0.04 Hz, spectral power for low frequency 0.04 to 0.15 Hz, spectral power for high frequency 0.15 to 0.4 Hz, features of the Poincaré plot-standard deviation of the small and large sub-axes of the Poincaré ellipse, their ratio and Poincaré ellipse volume, entropy of NN intervals, alpha value of the short-term fluctuations from detrended fluctuation analysis, alpha value of the long-term fluctuations from detrended fluctuation analysis, area under the curve of a single pulse;

Time series properties, catch 22—mode of z-scored distribution, 5-bin histogram, mode of z-scored distribution, 10-bin histogram, longest period of successive values above the average of the entire time series, time interval between successive extremes above the average, time interval between successive extremes below the average, time of the first transition below the value of 1/e in the autocorrelation function, first minimum of the autocorrelation function, total power of the lowest 5 frequencies in the Fourier spectrum, centroid of the complete Fourier spectrum, mean error in forecasting the next value of the time series from the average of the previous 3 values, time reversibility index, automutual information the value of mutual information between the time series and its copy, the first minimum of the automutual information function, the ratio of differences of successive points that are greater than 4% of the standard deviation of the entire series, longest period of successive decreases in signal values, entropy of two successive letters in the 3-letter symbolization of the time series, difference in correlation length between the original time series and the rescaled time series, which arises as the differences between each pair of successive values, exponential curve fit to successive distances in 2-d embedding space which originates by embedding points according to the time delay, ratio of slow fluctuations to faster ones, by linear detrending in each window, where the time series itself was downsampled to 50% of the original sampling frequency, ratio of slow fluctuations to faster ones, by linear detrending in each window in the original time series, trace of covariance matrix of transitions in a 3-letter symbolization of the time series, periodicity index.

The PPG signal is further analyzed by statistical methods and/or advanced machine learning methods, which classify the change of the above obtained parameters, one, more or their combination, for elevated or non-elevated filling pressures, and/or estimated, by regression, value of the filling pressure on the basis of this change or changes. The ability of classification and regression is obtained by training on a large volume of data from users with normal and abnormal filling pressures of the left ventricle of the heart.

The parameters are analyzed during the entire measurement and/or individually within one and the second position of the user and their change between the first and second position of the user.

Finally, information about the filling pressure of the left ventricle of the heart, from the analyzed photoplethysmographic signal, is displayed or interpreted to the user via a user interface of the device.

The present invention also provides a device for measuring, processing and analyzing the PPG signal for the non-invasive determination of the filling pressure of the left ventricle of the heart comprising a sensor of the PPG signal connected or connectable to a device containing a processor for executing program instructions for processing and analyzing the sensed PPG signal by the method according to the present invention, and a user interface for displaying or interpreting the results of the processing and analyzing the signal through this user interface.

The sensor of the PPG signal, the device containing the processor for executing program instructions for processing and analyzing the sensed PPG signal by the method according to the present invention and a user interface can be physically arranged in different assemblies, as one compact device containing all the mentioned members, or as an assembly of interconnected and/or connectable mentioned members.

The device for measuring, processing and analyzing the PPG signal for the non-invasive determination of the filling pressure of the left ventricle of the heart may also advantageously be provided in an arrangement comprising a sensor of the PPG signal connected or connectable to a device containing means for transmitting a PPG signal sensed by a sensor to a device containing a processor for executing program instructions for processing and analyzing the PPG signal by the method according to the present invention, means for receiving the results of the processing and analyzing the PPG signal from a device containing a processor, and a user interface for displaying or interpreting these results of the processing and analyzing the PPG signal.

For all possible arrangements of the device according to the present invention, the above stated processor is connected to a computer readable medium which is programmed with the steps for carrying out the above stated method and the device is adapted to carry out the above stated method in the steps programmed on the computer readable medium.

The solution is based on the different change of the PPG signal in users with normal and abnormal filling pressure of the left ventricle of the heart, which allows the use of commonly available devices without the need for any specialized equipment. From the user's point of view, only a very simple and undemanding maneuver is used to induce a change in blood volumes, which is easily reproducible, without inter-individual and intra-individual variability.

As set out above the portion of this aspect associated with receiving and processing the PPG comprises a method of measuring, processing and analyzing the photoplethysmographic signal for the non-invasive determination of the filling pressure of the left ventricle of the heart, whereby this method includes the steps: Receiving a photoplethysmographic signal, whereby the signal is either i) continuously sensed before, during and after a change in the user's position, or ii) sensed separately in a first position of the user and sensed separately in a second position of the user after the change of the position; Optionally Denoising and filtering of the PPG signal to specific frequencies;

Processing a photoplethysmographic signal, whereby this includes the following steps in the mentioned, or in a different order: Identification of individual heart beats;

Identification of the parts of the photoplethysmographic signal curve to obtain the area under the curve of a single pulse; Analysing of the photoplethysmographic signal by statistical methods and/or advanced machine learning methods, which classify the change of the area under the curve for elevated or non-elevated filling pressures, and/or estimated, by regression, value of the filling pressure on the basis of this change or changes, whereby the ability of classification and regression is obtained by training on a large volume of data from users with normal and abnormal filling pressures of the left ventricle of the heart, whereby the parameters are analyzed from the entire measurement and/or individually within one and the second position of the user, whereby for the evaluation of the filling pressure of the left ventricle of the heart a comparison between the first and second position is used.

This may further be expressed as a method of measuring, processing and analyzing the photoplethysmographic signal for the non-invasive determination of the filling pressure of the left ventricle of the heart, whereby this method includes the steps: Receiving a photoplethysmographic signal, whereby the signal is either i) continuously sensed before, during and after a change in the user's position, or ii) sensed separately in a first position of the user and sensed separately in a second position of the user after the change of the position; Processing a photoplethysmographic signal, whereby this includes:

Identification of individual heart beats; Obtaining the area under the curve of a single pulse associated with a heart beat; Evaluating the filling pressure of the left ventricle of the heart by comparing the area under the curve in the first position to the area under the curve in the second position.

In accordance with any preceding aspect the first signal and second signal each comprise a sequence of multiple pulses, and wherein determining the area under the curve comprises determining the area under the curve for each of the pulses of each signal, and then determining a central tendency for each signal, such as the median value for each signal.

A device such as a computational device comprising a processor and optionally a means of receiving data may perform the methods detailed above.

The device or method of any above aspect wherein a percentage difference in filling pressure or blood volume between two user positions of more than X categorizes a user as a first user type.

The device or method of any above aspect wherein a percentage difference in filling pressure or blood volume between two user positions of less than Y categorizes a user as a second type of user.

### BRIEF DESCRIPTION OF THE DRAWINGS

The invention is also explained with the help of the figures in the accompanying drawings, in which

FIG. 1 graphically illustrates the influence of active position change on hemodynamics in a user with heart failure and normal volume status, and a user with heart failure and volume overload;

FIG. 2 is an overview diagram of the PPG signal measurement and processing for the non-invasive determination of the filling pressure of the left ventricle of the heart, by means of the present invention;

FIG. 3 is a block diagram of an exemplary system for non-invasive assessment of pressure of the left ventricle of a user's heart according to the present invention;

FIG. 4 illustrates an example of an unprocessed PPG signal;

FIG. 5 graphically illustrates an example of PPG signal processing steps;

FIG. 6 graphically illustrates the course of the PPG signal curve processed by the method according to the present invention, before (on the left) and after (on the right) an active change of position in a user with heart failure with normal volume status;

FIG. 7 graphically illustrates the course of the PPG signal curve processed by the method according to the present invention, before (on the left) and after (on the right) an active change of position in a user with heart failure with volume overload.

FIG. 8 is a flow diagram illustrating one embodiment.

### DETAILED DESCRIPTION

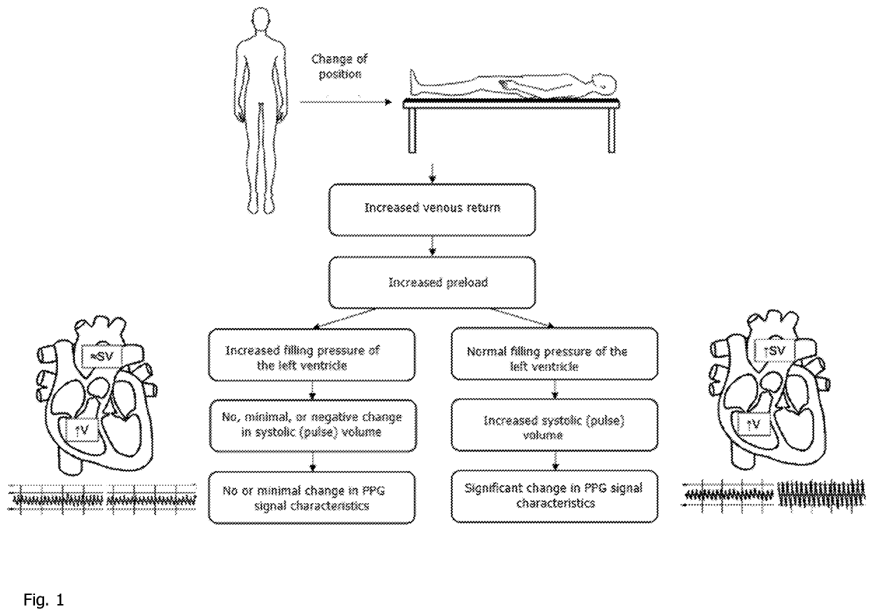

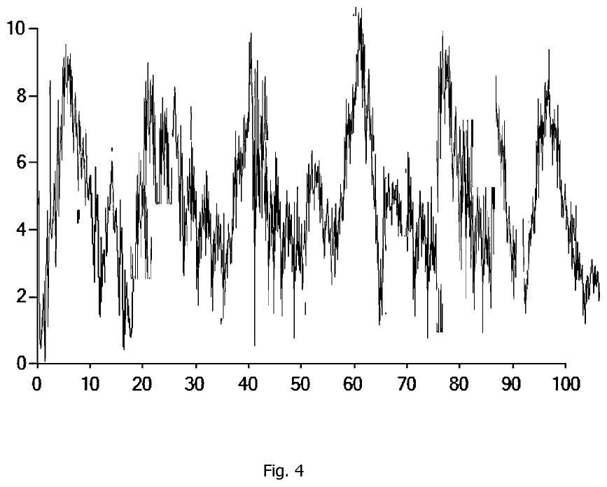

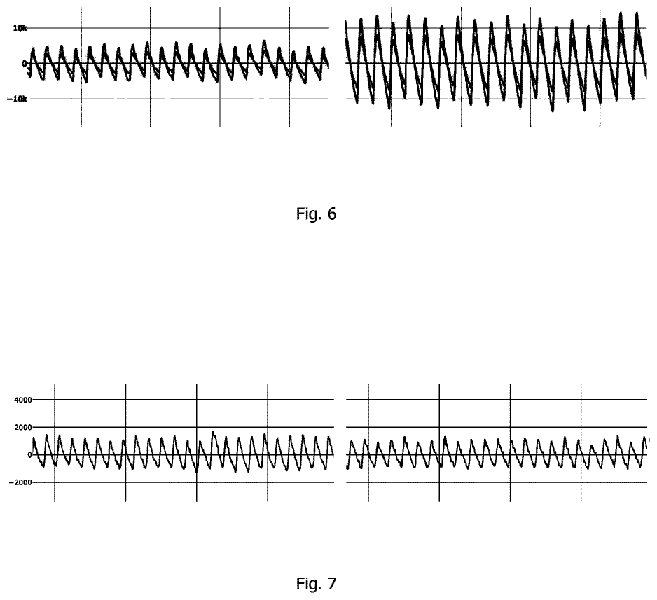

The mentioned invention will be further described in detail with reference to the exemplary embodiments in FIG. 1 to 7 in the accompanying drawings.

For the purposes of the present invention, any sensor 1 of the PPG signal, or device for measuring of the PPG signal can be used which senses or senses and records a PPG signal using either a transmission or a reflection method. The sensor of the PPG signal is understood as an element whose function is only sensing the PPG signal, and a device for measuring of the PPG signal is a complex device that includes a PPG sensor and beside sensing of the PPG signal has also other functions as e.g. recording of the PPG signal.

For the purposes of the present invention, the user interface of the device is understood as hardware as well as software elements of the device enabling the interaction of the device with the user, such as e.g. display, speakers, microphone, physical or software controls, etc.

A change in the user's position that triggers a hemodynamic change, or a change which leads to an increased venous return of blood to the heart, is understood in such a way that it can include both a total change of the user's position, i.e. a change of position from standing to sitting, from standing to lying down, from sitting to lying down, a total change of position on the bed, etc., as well as a non-total change of position, i.e. change of position of, for example, only the user's limb, e.g. lifting of the arm, the leg, or only the torso, etc.

The change of user's position for the purposes of the present invention may be thereby both active, where the user changes position by himself or herself without help, and passive, where the user is positioned with external assistance.

In general, the influence of a change of position, for example an active change of position, on hemodynamics in a user with heart failure and normal volume status, and a user with heart failure and volume overload is graphically explained in FIG. 1.

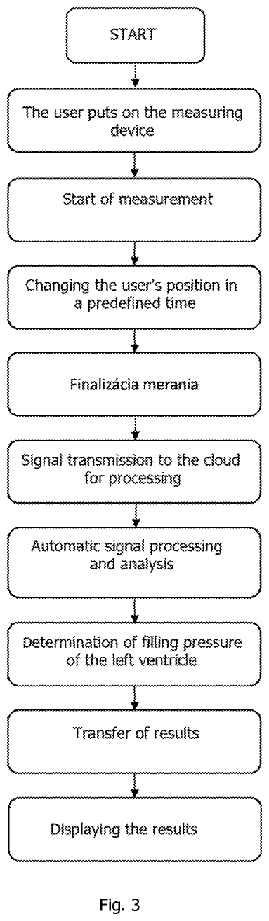

With reference to the block diagram mentioned in FIG. 3, an example of a system for non-invasive assessment of pressure of the left ventricle of a user's heart according to the present invention will be further described.

The user puts on a measuring device, in this example a sensor 1 of the PPG signal. The sensor 1 of the PPG can be practically placed anywhere on the user's body where a PPG signal can be sensed. This represents a universal solution for recording the PPG signal from multiple locations.

After the sensor 1 of the PPG signal is deployed, the actual measurement of the PPG signal starts. Preferably, the user is informed about the start of the measurement. The information about the start of the measurement may be preferably conveyed by written or spoken instructions, played via a user interface of the device, to which the PPG sensor 1 is connected or connectable. Such a device is preferably, for example, a smartphone 2 of the user on which the corresponding mobile application is operable. The sensor 1 of the PPG signal may be connected to the smartphone 2 wirelessly, e.g. via Bluetooth, Wi-fi, etc., or cable.

Via the user interface of the smartphone 2, any additional written or spoken instructions related to the measurement of the PPG signal are also preferably conveyed, for example regarding the time in which the user should actively change the position, end the measurement, etc.

After starting the measurement, the user receives an instruction for the above stated change of position, which leads to an increased venous return of blood to the heart, whereby the PPG signal is sensed continuously before, during and after changing the user's position, and/or separately in the first position of the user and separately in the second position of the user after the change of the position.

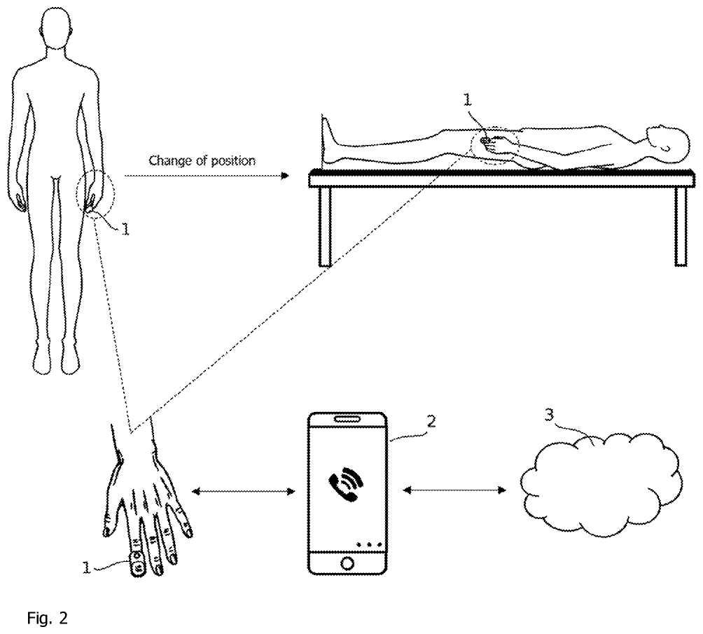

After the end of the measurement, the PPG signal obtained from the mentioned measurement is sent via smartphone 2 for processing, analysis and determination of the filling pressure of the left ventricle of the heart. Processing, analysis and determination of the filling pressure of the left ventricle of the heart in the embodiment shown in FIG. 2 takes place preferably through computing means in the cloud 3. The relevant results are then sent to user's smartphone 2, where they are interpreted via its user interface, i.e. e.g. typically displayed on the display of the device, or communicated vocally via the speakers of the device, or both.

In general, in the arrangement according to FIG. 2, the system is created in such a way that the sensor 1 of the PPG signal is connectable to a device which comprises a user interface. This device further comprises means for transmitting the sensed PPG signal to a device containing a processor for executing program instructions for processing and analyzing the PPG signal. The mentioned device comprising a user interface also preferably then comprises means for receiving the results of the processing and analyzing the PPG signal from a device containing a processor, i.e. in this case from the cloud 3. The user interface of the device then serves for displaying or interpreting the results of the processing and analyzing the signal to the user.

It is also possible, if a device with a user interface, i.e. for example the mentioned smartphone 2, has sufficient computing power, the PPG signal obtained from the sensor 1 may be processed and analyzed directly on this device with a user interface, whereby the results of the processing and analysis of the PPG signal are then respectively interpreted via a user interface of the device. With regard to the sensor 1 of the PPG signal, embodiments are also possible, where sensor 1 of the PPG signal is to the device with a user interface connected or connectable externally, or the sensor 1 of the PPG signal is directly part of the device with a user interface.

The PPG signal sensed by sensor 1 is subjected to denoising and filtering to specific frequencies.

The denoised and filtered PPG signal is evaluated using one, more or a combination of signal quality indices selected from the group-asymmetry coefficient, kurtosis, signal-to-noise ratio, entropy, rate of crossings through the zero axis, relative spectral power, peak value of the autocorrelation function, zero crossing of the autocorrelation function, spectral entropy, all of which are calculated on the time series as a whole and then on individual pulses and cumulatively, to determine whether the quality of the PPG signal is sufficient for subsequent processing and analysis.

In the event that the signal quality is not sufficient, the system will recommend the user to repeat the measurement, i.e. from the “START” position in FIG. 3, since the results of processing a poor-quality signal could be incorrect-false positive or negative. Due to the fact that this system does not contain any specialized devices, and does not contain maneuvers that are relatively difficult to repeat accurately, such as the Valsalva maneuver, restarting the measurement and performing the subsequent steps of the method is undemanding and unburdening to the user.

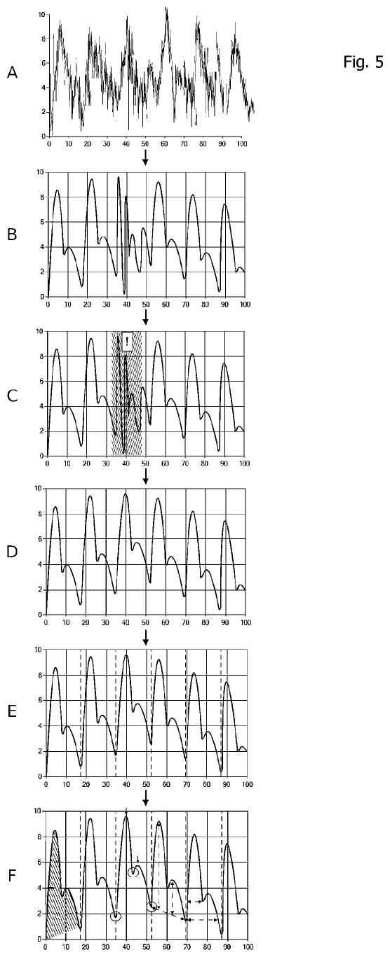

Subsequently, the PPG signal processing is carried out in the following steps, graphically represented in FIG. 5, in the below stated, or in a different order; the selection and order of the processing steps and their parameters are specific to the device—selected by testing the optimal configuration for a specific measuring device:
Identification of artifacts;
Identification of individual heart beats;
Identification of the parts of the curve of the PPG signal to obtain one, more or a combination of parameters selected from the below stated group or groups (for the sake of accuracy, some terms are assigned their English equivalent in brackets):
Physiological parameters—blood oxygen saturation, heart rate calculated from individual pulses or from Empirical decomposition into modes, respiratory rate calculated from individual pulses—XB123EF1FM1 algorithm or from empirical decomposition into modes;

Pulse wave characteristics—systolic peak position, systolic peak value, diastolic peak position, diastolic peak value, position of the lowest point on the wave, value of the lowest point on the wave, amplitude (value of the highest point on the wave), position of the highest point on the wave (in English Crest time), maximum slope, systolic length expressed in percentiles for percentiles [0.1, 0.25, 0.33, 0.5, 0.66] (in English Systolic length percentiles [0.1, 0.25, 0.33, 0.5, 0.66]), index B/A (in English B over A) whereby B is the value of the minimum of the second derivative of the PPG signal and A is the value of the maximum of the second derivative of the PPG signal, diastolic length expressed in percentiles for percentiles [0.1, 0.25, 0.33, 0.5, 0.66] (in English Diastolic length percentiles [0.1, 0.25, 0.33, 0.5, 0.66]), average, minimum and maximum of NN intervals (in English NN intervals—NN interval is the time between individual pulses, in the event that the whole signal contains 100 pulses, we can count 99 NN intervals), minimum and maximum of differences in NN intervals (in English NN intervals difference-from a series of NN intervals we calculate their difference, in the event that the whole signal contains 100 pulses, we have 99 NN intervals and 98 differences in NN intervals), standard deviation of NN intervals (in English SDNN—standard deviation of NN interval), the average of the standard deviations of all NN intervals for each 5-minute segment in a long recording (in English SDNN index), the standard deviation of the averages of NN intervals for each 5-minute segment in a long recording (in English SDANN standard deviation of the average NN interval), root mean square value of differences in NN intervals (in English RMSNN-root mean square of NN interval differences), the number of NN intervals over 20, resp. 50 ms, spectral power for very low frequency up to 0.04 Hz, spectral power for low frequency 0.04 to 0.15 Hz, spectral power for high frequency 0.15 to 0.4 Hz, features of the Poincaré plot (in English features of the Poincaré plot)-standard deviation of the small and large sub-axes of the Poincaré ellipse, their ratio and Poincaré ellipse volume, entropy of NN intervals (in English sample entropy of the NN series), alpha value of the short-term fluctuations from detrended fluctuation analysis (in English alpha value of the short-term fluctuations from DFA-detrended fluctuation analysis), alpha value of the long-term fluctuations from detrended fluctuation analysis (in English alpha value of the long-term fluctuations from DFA-detrended fluctuation analysis), area under the curve of a single pulse;

Time series properties, catch 22—mode of z-scored distribution, 5-bin histogram (in English Mode of z-scored distribution, 5-bin histogram), mode of z-scored distribution, 10-bin histogram (in English Mode of z-scored distribution, 10-bin histogram), longest period of successive values above the average of the entire time series, time interval between successive extremes above the average, time interval between successive extremes below the average, time of the first transition below the value 1/e in the autocorrelation function, the first minimum of the autocorrelation function, total power of the lowest 5 frequencies in the Fourier spectrum, centroid of the complete Fourier spectrum, mean error in forecasting the next value of the time series from the average of the previous 3 values (in English Mean error from a rolling 3-sample mean forecasting), time reversibility index-it is calculated as the average of the third powers of the differences of successive points (in English Time-reversibility statistic), automutual information (in English Automutual information)—value of the mutual information between the time series and its copy, the first minimum of the automutual information function, the ratio of differences of successive points that are greater than 4% of the standard deviation of the entire series (in English Proportion of successive differences exceeding 0.04σ), longest period of successive decreases in signal values, entropy of two successive letters in the 3-letter symbolization of the time series (in English Shannon entropy of two successive letters in equiprobable 3-letter symbolization), difference in correlation length between the original time series and the rescaled time series, which arises as the differences between each pair of successive values (in English Change in correlation length after iterative differencing), exponential curve fit to successive distances in 2-d embedding space which originates by embedding points according to the time delay (in English Exponential fit to successive distances in 2-d embedding space), ratio of slow fluctuations to faster ones, by linear detrending in each window, where the time series itself was downsampled to 50% of the original sampling frequency (in English Proportion of slower timescale fluctuations that scale with DFA, 50% sampling), ratio of slow fluctuations to faster ones, by linear detrending in each window in the original time series (in English Proportion of slower timescale fluctuations that scale with linearly rescaled range fits), trace of covariance matrix of transitions in a 3-letter symbolization of the time series (in English Trace of covariance of transition matrix between symbols in 3-letter alphabet), periodicity index according to Wang et al. 2007.

For completeness, periodicity index according to Wang et al. 2007 refers to Wang X, Wirth A, Wang L (2007) Structure-based statistical features and multivariate time series clustering. In: Proceedings—IEEE international conference on data mining, ICDM, pp 351-360. ISSN 15504786. https://doi.org/ 10.1109/ICDM.2007.103

In FIG. 5, the corresponding graphical representations of the sequentially processed PPG signal are shown for the mentioned steps, where graph A represents the raw PPG signal, graph B represents the signal denoised and filtered to specific frequencies, graph C represents the signal with the artifact detected, graph D represents the signal with the artifact removed, graph E represents the signal with the individual heartbeats detected, and graph F contains some selected signal parameters marked for analysis.

The PPG signal is further analyzed by statistical methods, both Bayesian and frequentist, and/or advanced machine learning methods, which classify the change of the above obtained parameters, one, more or their combination, for elevated or non-elevated filling pressures, and/or estimated, by regression, value of the filling pressure on the basis of this change or changes. The ability of classification and regression is obtained by training on a large volume of data from users with normal and abnormal filling pressures of the left ventricle of the heart.

The parameters are analyzed from the entire measurement and/or individually within one and the second position of the user, whereby for the evaluation of the filling pressure of the left ventricle of the heart a comparison between the first and second position is used, i.e. the comparison between the above stated parameters of the PPG signal obtained from the measurement of the PPG signal in the first position of the user, i.e. before the change of the position of the user, and the above stated parameters of the PPG signal obtained from the measurement of the PPG signal in the second position of the user, i.e. in the position after the change of the position of the user?.

From the mentioned analysis of the parameters, the filling pressure of the left ventricle of the heart is determined—reduced, normal, increased or its specific value.

Information about the filling pressure of the left ventricle of the heart is then displayed or otherwise interpreted, e.g. by voice, to the user via a user interface. With regard to the information itself about the filling pressure of the left ventricle of the heart, this can be in different forms, e.g. as a specific numerical value, or as a relative figure, e.g. “reduced”, “normal”, “increased”, etc.

The device for measuring, processing and analyzing the PPG signal for the non-invasive determination of the filling pressure of the left ventricle of the heart, according to the present invention, i.e. for carrying out the method and steps described above, will comprise, in a most advantageous embodiment for the user, a sensor 1 of the PPG signal preferably wirelessly connectable to a smartphone 2. The sensor 1 of the PPG signal may also be connected to the smartphone 2 via a cable. From the smartphone 2, the PPG signal is typically transmitted over the internet to the cloud 3, which contains computing means for the above stated processing and analysis of the sensed PPG signal and sending the results from the processing and analysis of the PPG signal from the cloud 3 back to the user's smartphone 2. The results from the processing and analysis will then be displayed or otherwise interpreted via a user interface of the smartphone 2. The mentioned functions of the smartphone 2 will typically be provided by a dedicated mobile application.

The device for measuring, processing and analyzing the PPG signal for the non-invasive determination of the filling pressure of the left ventricle of the heart may be provided generally in an arrangement which contains a sensor 1 of the PPG signal connected or connectable to a device containing a processor for executing program instructions for processing and analyzing the sensed PPG signal by the above stated method according to the present invention, and a device with a user interface for displaying or interpreting the results of the processing and analyzing the signal through this user interface.

The sensor 1 of the PPG signal, the device containing the processor for executing program instructions for processing and analyzing the sensed PPG signal by the method according to the present invention and a device with a user interface can be physically arranged in different assemblies, as one compact device containing all the mentioned members, or as an assembly of interconnected and/or connectable mentioned members.

In one arrangement, the device for measuring, processing and analyzing the PPG signal for the non-invasive determination of the filling pressure of the left ventricle of the heart can therefore be provided in an arrangement that contains a sensor of the PPG signal connected or connectable to a device containing means for transmitting a PPG signal sensed by a sensor to a remote device containing a processor for executing program instructions for processing and analyzing the PPG signal by the method according to the present invention, means for receiving the results of the processing and analyzing the PPG signal from the remote device containing a processor, and a user interface for displaying or interpreting these results of the processing and analyzing the PPG signal. Such a device is the most advantageous from the user's point of view e.g. smartphone 2 and a remote device containing a processor, e.g. a cloud 3, a local PC or server, etc.

In another arrangement, the device containing the processor for executing program instructions for processing and analyzing the PPG signal by the method according to the present invention may at the same time be a device that directly receives PPG signal from the sensor 1, whereby this device comprises at the same time also a user interface for displaying or interpreting the results of the processing and analyzing the signal through this user interface, which may be, for example, a smartphone 2 itself, a tablet, a local PC, etc.

For all possible arrangements of the device according to the present invention, the above stated processor is connected to a computer readable medium which is programmed with the steps for carrying out the above stated method and the device is adapted to carry out the above stated method in the steps programmed on the computer readable medium.

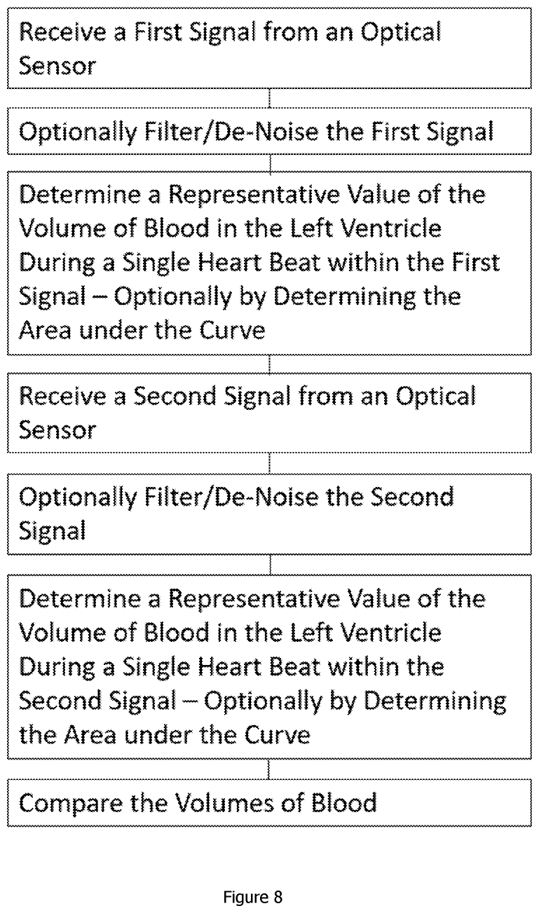

FIG. 8 shows a method in accordance with one embodiment. This embodiment focuses on the steps performed by a device for analyzing a PPG signal. It is noted that this signal may instead be an equivalent signal, such as any optical signal related to the monitoring of the cardiovascular system. Whilst it is set-out above and in the claims that this device may be part of a system comprising a sensor, and/or a user interface device these elements are not discussed herein and are considered optional.

FIG. 5 shows a series of graphs illustrating PPG sensor data. The X-axis is time (and may be any equivalent metric such as number of light pulses etc.). The Y axis is an indication of the amount of blood in the vessels being monitored. For example, where the PPG is taken from a finger this may correlate to the amount of blood in the finger capillaries. Any equivalent measure may be used as the Y-axis. In the present examples the Y-axis is a direct measurement of the amount of light received by the sensor. This is an indication of the amount of blood in the relevant vessels being monitored.

Graph A shows the raw data recorded by the sensor. This may be the data received by the device. This raw data may be analyzed directly. For example, the raw data may be reviewed and a single period with the lowest amount of noise and/or distortion may be identified. This may be determined for example by assessing which of the pulses corresponds most closely to a known archetypal pulse shape. This determination may be performed by any suitable means such as shape/fingerprint correlation or the like with the pulse with highest correlation to an archetypal shape being selected. This pulse may then be used for analysis. In this case, the optional step filtering the first signal and/or the second signal is not needed.

Alternatively, Graph B Shows a De-noised Version of Graph a. This Data May be used.

Alternatively, the data is graphs D, E or F may be used as in these graphs artefacts have been removed, and the data further smoothed and/or corrected.

A signal may comprise any number of pulses (referring to a heartbeat comprising a period of systole and a period of diastole). The signal may be continuous or more likely will comprise two distinct signals. The first distinct signal is a signal measured when the user is in a first position (e.g. the user is standing vertically upright). The second distinct signal is a signal when the user is in a second position (e.g. the user is laying horizontally down). Where the signal is continuous the signal may include both portions, and therefore may be broken down into sections corresponding to these distinct signals.

The determination of the filling pressure of the left ventricle of the heart, or of the volume of blood in the left ventricle during a pulse, may then be performed using this data. This may be achieved by determining the area under the curve of a single pulse. In this case the curve is the shape formed in the PPG signal by a single period of one heartbeat comprising systole and diastole.

Where the first signal comprises a single period this may be performed through any means of integration-such as numerical integration. The same may then be performed for the second signal. The signals may comprise a single period in a number of embodiments-for example when one period is selected from raw data as illustrating the lowest amount of noise/artefacts. These values can then be compared with each other.

It is noted that the area under the curve of a single pulse in the graph is a relative measurement. This correlates to one or both of the filling pressure of the left ventricle of the heart, and/or to the volume of blood in the left ventricle during a pulse. However, this is not a direct measurement of either value, and is not calibrated to achieve an absolute specific volume or absolute specific pressure. Rather this relative number is useful for comparing the volume of blood/filling pressure in the left ventricle as the user is in multiple positions- and it is not necessary to determine an absolute value in this case. It is of course possible to calibrate the system such than an absolute value is determined-but this is not essential to the inventive concept.

Where the first signal and the second signal each comprise a plurality of pulses the area under the curve may be determined in one of several different ways. In one embodiment the entire signal comprising a plurality of pulses is integrated. This determines the area under the curve either i) for the whole signal, or ii) for each of the pulses within the signal. In the first embodiment where the integration determines the area under the curve for the whole signal then if the first signal and the second signal comprise the same number of the pulses then this total value of area may be directly compared. Where the first signal and second signal comprise different number of pulses this may be accounted for, for example by dividing both signals by the number of pulses they comprise to determine an average (mean) area under the curve for each signal. This can then be compared with the second signal. For the second embodiment each signal will comprise a series of pulses, and each pulse will have an associated area under the curve. Any pulse may be selected and compared to the corresponding pulse of the other signal. Alternatively, the areas of each pulse of each signal may be ordered. Outlying values may then optionally be discarded. A measure of central tendency, such as the median area may then be found for each of the signals. This median area under the curve may then be compared to the other signal. In some embodiments a modal average may be used rather than a median. This may be the case where the areas under the curve are categorized into which of several intervals they fall into (e.g. 7.5-8.0 if the typical value is between 5 and 10) and then the modal interval may be selected. This may be less precise but more reliable. Measures of central tendency include but are not limited to: median, mean, mode, trimean and others.

When comparing the values of the area under the curve of the first signal and the second signal a percentage difference between the two values may be determined. This may be interpreted either as a difference in filling pressure expressed in mmHg or as a volume of blood expressed in ml.

The difference may be compared to a threshold. For example, if the percentage difference is more than 100% (or 50% if the order of the positions is revered—the other values below will be similarly affected if the order of positions is reversed) then the user may be categorized as being a first type of user. In a more preferable embodiment this threshold may be 150% as this has been found to be optimal for this categorization. If the percentage difference is less than 85% then the user may be categorized as a second type of user. More preferably this threshold may be 60%, and most preferably 50%. The first type of user may be considered healthy and the second type of user may be considered abnormal. Users with a result between the thresholds may remain uncategorized or the test may be re-performed, or an additional test performed.

### Industrial applicability

The present invention provides a widely available and non-invasive solution for monitoring the filling pressure of the left ventricle of the heart in users with heart failure, both diagnosed and not yet diagnosed; thus, the invention enables the diagnosis of heart failure in undiagnosed users and monitoring the state of heart failure in users with already diagnosed heart failure. This invention can be used, for example, for home and clinical monitoring, therapy optimization, and also as a measuring device for further heart failure research in multiple fields including physiology, pathophysiology, pharmacology and clinical practice.

1. A method of measuring, processing and analyzing the photoplethysmographic signal for the non-invasive determination of the filling pressure of the left ventricle of the heart using a sensor (1) of the photoplethysmographic signal connected or connectable to a device containing a processor for executing program instructions for processing the sensed photoplethysmographic signal and a user interface for displaying or interpreting the results of the processing and analyzing the photoplethysmographic signal via a user interface, or using a sensor (1) of the photoplethysmographic signal connected or connectable to a device containing means for transmitting of the sensed photoplethysmographic signal to a remote device containing a processor for executing program instructions for processing and analyzing the photoplethysmographic signal, means for receiving the results of the processing and analyzing the photoplethysmographic signal from the remote device containing a processor, and a user interface for displaying or interpreting the results of the processing and analyzing the photoplethysmographic signal via a user interface, whereby this method includes the steps:
Sensing the photoplethysmographic signal by a photoplethysmographic signal sensor or by a device for sensing a photoplethysmographic signal during a passive or active change in the position of the user, which leads to an increased venous return of blood to the heart, whereby the signal is continuously sensed before, during and after changing the user's position, or separately in the first position of the user and separately in the second position of the user after the change of the position;
Denoising and filtering of the PPG signal to specific frequencies;
Evaluation of denoised and filtered photoplethysmographic signal using one, more or a combination of signal quality indices selected from the group comprising the asymmetry coefficient, kurtosis, signal-to-noise ratio, entropy, rate of crossings through the zero axis, relative spectral power, peak value of the autocorrelation function, zero crossing of the autocorrelation function, spectral entropy, all of which are calculated on the time series as a whole and then on individual pulses and cumulatively to determine the quality of the photoplethysmographic signal sufficient for the determination of the filling pressure of the left ventricle of the heart;
Processing a photoplethysmographic signal, whereby this includes the following steps in the mentioned, or in a different order:
Identification of artifacts;
Identification of individual heart beats;
Identification of the parts of the photoplethysmographic signal curve to obtain one, more or a combination of parameters selected from the group comprising the
Physiological parameters: blood oxygen saturation, heart rate calculated from individual pulses or from Empirical decomposition into modes, respiratory rate calculated from individual pulses XB123EF1FM1 algorithm or from empirical decomposition into modes,
Pulse wave characteristics: systolic peak position, systolic peak value, diastolic peak position, diastolic peak value, position of the lowest point on the wave, value of the lowest point on the wave, amplitude (value of the highest point on the wave), position of the highest point on the wave, maximum slope, systolic length expressed in percentiles for percentiles [0.1, 0.25, 0.33, 0.5, 0.66], index B/A, whereby B is the value of the minimum of the second derivative of the PPG signal and A is the value of the maximum of the second derivative of the PPG signal, diastolic length expressed in percentiles for percentiles [0.1, 0.25, 0.33, 0.5, 0.66], the average, minimum and maximum of the NN intervals, the minimum and maximum of the differences in the NN intervals, the standard deviation of the NN intervals, average of the standard deviations of all NN intervals for each 5-minute segment in the long recording, the standard deviation of the averages of NN intervals for each 5-minute segment in a long recording, the root mean square value of the differences in NN intervals, the number of NN intervals over 20, resp. 50 ms, spectral power for very low frequency up to 0.04 Hz, spectral power for low frequency 0.04 to 0.15 Hz, spectral power for high frequency 0.15 to 0.4 Hz, features of the Poincaré plot-standard deviation of the small and large sub-axes of the Poincaré ellipse, their ratio and Poincaré ellipse volume, entropy of NN intervals, alpha value of the short-term fluctuations from detrended fluctuation analysis, alpha value of the long-term fluctuations from detrended fluctuation analysis, area under the curve of a single pulse;
Time series properties, catch 22: mode of z-scored distribution, 5-bin histogram, mode of z-scored distribution, 10-bin histogram, longest period of successive values above the average of the entire time series, time interval between successive extremes above the average, time interval between successive extremes below the average, time of the first transition below the value of 1/e in the autocorrelation function, first minimum of the autocorrelation function, total power of the lowest 5 frequencies in the Fourier spectrum, centroid of the complete Fourier spectrum, mean error in forecasting the next value of the time series from the average of the previous 3 values, time reversibility index, automutual information-the value of mutual information between the time series and its copy, the first minimum of the automutual information function, the ratio of differences of successive points that are greater than 4% of the standard deviation of the entire series, longest period of successive decreases in signal values, entropy of two successive letters in the 3-letter symbolization of the time series, difference in correlation length between the original time series and the rescaled time series, which arises as the differences between each pair of successive values, exponential curve fit to successive distances in 2-d embedding space which originates by embedding points according to the time delay, ratio of slow fluctuations to faster ones, by linear detrending in each window, where the time series itself was downsampled to 50% of the original sampling frequency, ratio of slow fluctuations to faster ones, by linear detrending in each window in the original time series, trace of covariance matrix of transitions in a 3-letter symbolization of the time series, periodicity index;
Analysis of the photoplethysmographic signal by statistical methods and/or advanced machine learning methods, which classify the change of the above obtained parameters, one, more or their combination, for elevated or non-elevated filling pressures, and/or estimated, by regression, value of the filling pressure on the basis of this change or changes, whereby the ability of classification and regression is obtained by training on a large volume of data from users with normal and abnormal filling pressures of the left ventricle of the heart, whereby the parameters are analyzed from the entire measurement and/or individually within one and the second position of the user, whereby for the evaluation of the filling pressure of the left ventricle of the heart a comparison between the first and second position is used
Displaying or interpreting information about the filling pressure of the left ventricle of the heart, from the analyzed photoplethysmographic signal, to the user via the user interface of the device.

2. The device for measuring, processing and analyzing the photoplethysmographic signal for the non-invasive determination of the filling pressure of the left ventricle of the heart comprising a sensor (1) of the photoplethysmographic signal connected or connectable to a device containing a processor for executing program instructions for processing and analyzing the sensed photoplethysmographic signal and a user interface for displaying or interpreting the results of the processing and analyzing the photoplethysmographic signal via a user interface, or sensor (1) of the photoplethysmographic signal connected or connectable to a device containing means for transmitting the sensed photoplethysmographic signal to a remote device containing a processor for executing program instructions for processing and analyzing the photoplethysmographic signal, means for receiving the results of the processing and analyzing the photoplethysmographic signal from the remote device containing a processor, and a user interface for displaying or interpreting the results of the processing and analyzing the photoplethysmographic signal via a user interface, whereby the processor is connected to a computer-readable medium, whereby the computer-readable medium is programmed with steps and the device is adapted to perform the method steps programmed on the computer-readable medium, wherein the method steps include:
Sensing the photoplethysmographic signal by a photoplethysmographic signal sensor or by a device for sensing a photoplethysmographic signal during a passive or active change in the position of the user, which leads to an increased venous return of blood to the heart, whereby the signal is continuously sensed before, during and after changing the user's position, or separately in the first position of the user and separately in the second position of the user after the change of the position;
Denoising and filtering a photoplethysmographic signal to specific frequencies;
Evaluation of denoised and filtered photoplethysmographic signal using one, more or a combination of signal quality indices selected from the group comprising the asymmetry coefficient, kurtosis, signal-to-noise ratio, entropy, rate of crossings through the zero axis, relative spectral power, peak value of the autocorrelation function, zero crossing of the autocorrelation function, spectral entropy, all of which are calculated on the time series as a whole and then on individual pulses and cumulatively to determine the quality of the photoplethysmographic signal sufficient for the determination of the filling pressure of the left ventricle of the heart;
Processing a photoplethysmographic signal, whereby this includes the following steps in the mentioned, or in a different order:
Identification of artifacts;
Identification of individual heart beats;
Identification of the parts of the photoplethysmographic signal curve to obtain one, more or a combination of parameters selected from the group comprising the
Physiological parameters: blood oxygen saturation, heart rate calculated from individual pulses or from Empirical decomposition into modes, respiratory rate calculated from individual pulses XB123EF1FM1 algorithm or from empirical decomposition into modes,
Pulse wave characteristics: systolic peak position, systolic peak value, diastolic peak position, diastolic peak value, position of the lowest point on the wave, value of the lowest point on the wave, amplitude (value of the highest point on the wave), position of the highest point on the wave, maximum slope, systolic length expressed in percentiles for percentiles [0.1, 0.25, 0.33, 0.5, 0.66], index B/A, whereby B is the value of the minimum of the second derivative of the PPG signal and A is the value of the maximum of the second derivative of the PPG signal, diastolic length expressed in percentiles for percentiles [0.1, 0.25, 0.33, 0.5, 0.66], the average, minimum and maximum of the NN intervals, the minimum and maximum of the differences in the NN intervals, the standard deviation of the NN intervals, average of the standard deviations of all NN intervals for each 5-minute segment in the long recording, the standard deviation of the averages of NN intervals for each 5-minute segment in a long recording, the root mean square value of the differences in NN intervals, the number of NN intervals over 20, resp. 50 ms, spectral power for very low frequency up to 0.04 Hz, spectral power for low frequency 0.04 to 0.15 Hz, spectral power for high frequency 0.15 to 0.4 Hz, features of the Poincaré plot-standard deviation of the small and large sub-axes of the Poincaré ellipse, their ratio and Poincaré ellipse volume, entropy of NN intervals, alpha value of the short-term fluctuations from detrended fluctuation analysis, alpha value of the long-term fluctuations from detrended fluctuation analysis, area under the curve of a single pulse;
Time series properties, catch 22: mode of z-scored distribution, 5-bin histogram, mode of z-scored distribution, 10-bin histogram, longest period of successive values above the average of the entire time series, time interval between successive extremes above the average, time interval between successive extremes below the average, time of the first transition below the value of 1/e in the autocorrelation function, first minimum of the autocorrelation function, total power of the lowest 5 frequencies in the Fourier spectrum, centroid of the complete Fourier spectrum, mean error in forecasting the next value of the time series from the average of the previous 3 values, time reversibility index, automutual information-the value of mutual information between the time series and its copy, the first minimum of the automutual information function, the ratio of differences of successive points that are greater than 4% of the standard deviation of the entire series, longest period of successive decreases in signal values, entropy of two successive letters in the 3-letter symbolization of the time series, difference in correlation length between the original time series and the rescaled time series, which arises as the differences between each pair of successive values, exponential curve fit to successive distances in 2-d embedding space which originates by embedding points according to the time delay, ratio of slow fluctuations to faster ones, by linear detrending in each window, where the time series itself was downsampled to 50% of the original sampling frequency, ratio of slow fluctuations to faster ones, by linear detrending in each window in the original time series, trace of covariance matrix of transitions in a 3-letter symbolization of the time series, periodicity index,
Analysis of the photoplethysmographic signal by statistical methods and/or advanced machine learning methods, which classify the change of the above obtained parameters, one, more or their combination, for elevated or non-elevated filling pressures, and/or estimated, by regression, value of the filling pressure on the basis of this change or changes, whereby the ability of classification and regression is obtained by training on a large volume of data from users with normal and abnormal filling pressures of the left ventricle of the heart, whereby the parameters are analyzed from the entire measurement and/or individually within one and the second position of the user, whereby for the evaluation of the filling pressure of the left ventricle of the heart a comparison between the first and second position is used;
Displaying or interpreting information about the filling pressure of the left ventricle of the heart, from the analyzed photoplethysmographic signal, to the user via the user interface of the device.
